(19) United States
(12) Patent Application Publication
(10) Pub. No.: US 2013/0173021 A1
(43) Pub. Date: Jul. 4, 2013

(54) KNEE JOINT

(71) Applicants: Chien-Wen CHEN, New Taipei City (TW); Chien-Cheng Chen, New Taipei City (TW)

(72) Inventors: Chien-Wen CHEN, New Taipei City (TW); Chien-Cheng Chen, New Taipei City (TW)

(21) Appl. No.: 13/611,124

(22) Filed: Sep. 12, 2012

(30) Foreign Application Priority Data
Dec. 29, 2011 (TW) 100224911

(51) Int. Cl. A61F 2/64

(52) U.S. Cl. 623/46

(57) ABSTRACT

A knee joint includes a knee member, an upper connecting member that is disposed on top of the knee member for connection with a prosthetic upper leg, a resilient member disposed between the knee member and the upper connecting member, and a connection seat that has an upper portion pivotally connected to the knee member and that has a lower portion for connecting with a prosthetic lower leg.

### CROSS-REFERENCE TO RELATED APPLICATION

This application claims priority of Taiwanese Application No. 100224911, filed on Dec. 29, 2011, the disclosure of which is incorporated herein by reference.

### BACKGROUND OF THE INVENTION

1. Field of the Invention

The invention relates to a knee joint, and more particularly to a prosthetic knee joint having a resilient member.

2. Description of the Related Art

Currently, an improvement on shock absorption effect of a prosthetic knee joint is focused on how to maintain a support to a stump limb of a physically disabled wearer when the prosthetic knee joint is bent, i.e., when walking on an uneven ground surface, the wearer can avoid being at the risk of falling due to loss of center of gravity of the body caused by a bend of the prosthetic knee joint.

Although the risk of falling encountered in the conventional prosthetic knee joint may be alleviated, there is still plenty of room for improvement in comfort. In particular, when the wearer wearing the knee joint walks and steps on the ground, the reaction force is directly transmitted to a stump limb of the wearer to result in the wearer discomfort. Hence, this invention is directed to how influence of the reaction force acting on the wearer when walking may be reduced.

### SUMMARY OF THE INVENTION

Therefore, an object of the present invention is to provide a knee joint having an improved shock-absorbing effect.

According to the present invention, a knee joint includes a knee member, an upper connecting member that is disposed on top of the knee member for connection with a prosthetic upper leg, a resilient member disposed between the knee member and the upper connecting member, and a connection seat that has an upper portion pivotally connected to the knee member and that has a lower portion for connecting with a prosthetic lower leg.

### BRIEF DESCRIPTION OF THE DRAWINGS

Other features and advantages of the present invention will become apparent in the following detailed description of the preferred embodiment with reference to the accompanying drawings, of which:

FIG. 1 is an exploded perspective view illustrating the preferred embodiment of a knee joint according to the present invention;

FIG. 2 is a side view illustrating a transmission path of a reaction force when the knee joint of the preferred embodiment of FIG. 1 is in use; and

FIG. 3 is a partly sectional view illustrating engagement relationships among an upper connecting member, a knee member and a resilient member of the knee joint of the preferred embodiment of FIG. 1.

### DETAILED DESCRIPTION OF THE PREFERRED EMBODIMENT

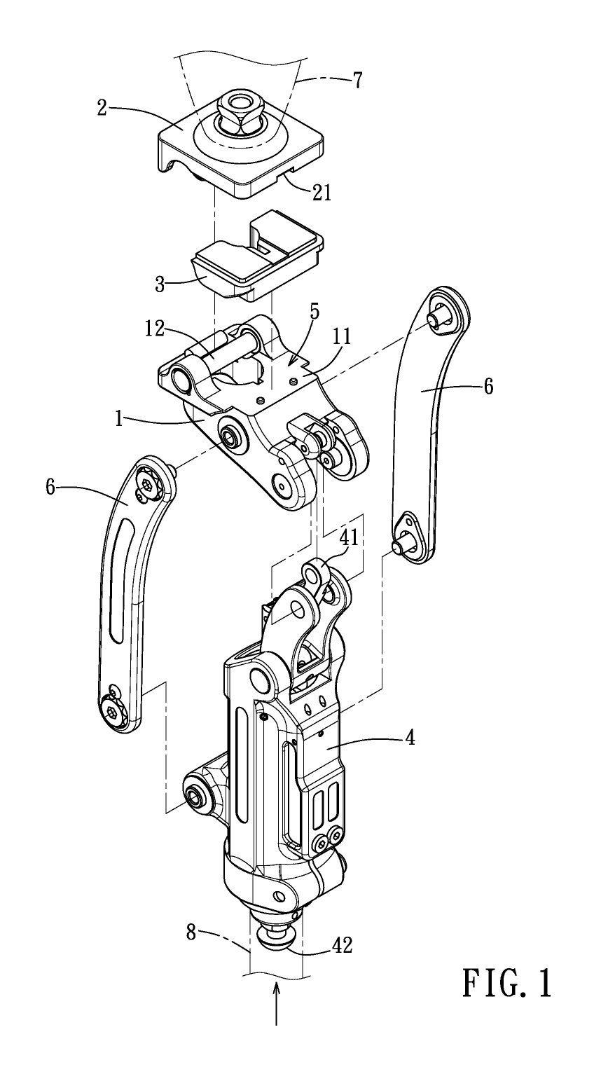

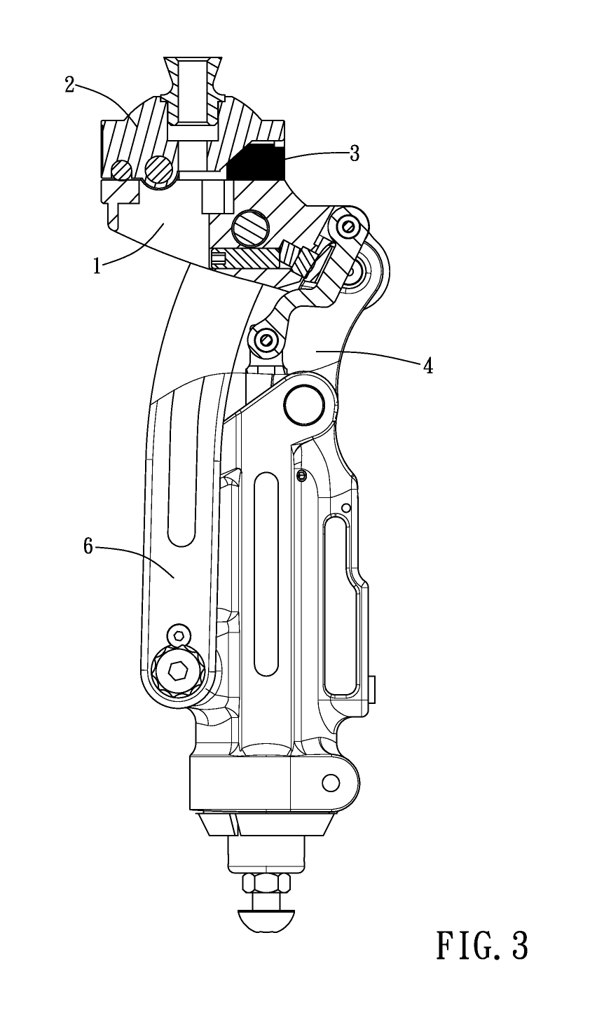

Referring to FIGS. 1 to 3, the preferred embodiment of a knee joint of the present invention adapted for connection with a prosthetic upper leg 7 and a prosthetic lower leg 8 is illustrated. The knee joint includes a knee member 1, an upper connecting member 2, a resilient member 3, and a connection seat 4.

The upper connecting member 2 is disposed on top of the knee member 1, is pivotally connected to the knee member 1, and is for connection with the prosthetic upper leg 7 . A bottom surface 21 of the upper connecting member 2 and an upper surface 11 of the knee member 1 cooperate to define a buffer space 5 therebetween.

Preferably, the knee member 1 has a pivot part 12 disposed rearwardly of the buffer space 5 and connected pivotally to the upper connecting member 2.

The resilient member 3 is disposed between the knee member 1 and the upper connecting member 2. Preferably, the resilient member 3 is disposed in the buffer space 5. The upper connecting member 2 and the knee member 1 compress the resilient member 3 when pivoting relative to each other.

In this embodiment, the resilient member 3 may be a rubber pad that is configured to conform to the bottom surface 21 of the upper connecting member 2 and the upper surface 11 of the knee member 1, and that is received fittingly in the buffer space 5.

The connection seat 4 has an upper portion 41 pivotally connected to the knee member 1, and a lower portion 42 for connecting with the prosthetic lower leg 8.

The knee joint further includes two spaced-apart arcuate plates 6. Each of the arcuate plates 6 has two opposite ends respectively and pivotally connected to the knee member 1 and the connection seat 4. The arcuate plates 6 are useful for confining the pivotal movement of the knee member 1 relative to the connection seat 4 and maintaining stability of the pivotal movement therebetween.

When a physically disabled wearer wears a prosthesis including the prosthetic upper leg 7, the prosthetic lower leg 8 and the knee joint of this invention connected therebetween and walks, a prosthetic foot (not shown) of the prosthetic lower leg 8 contacts the ground and receives a reaction force therefrom with every step.

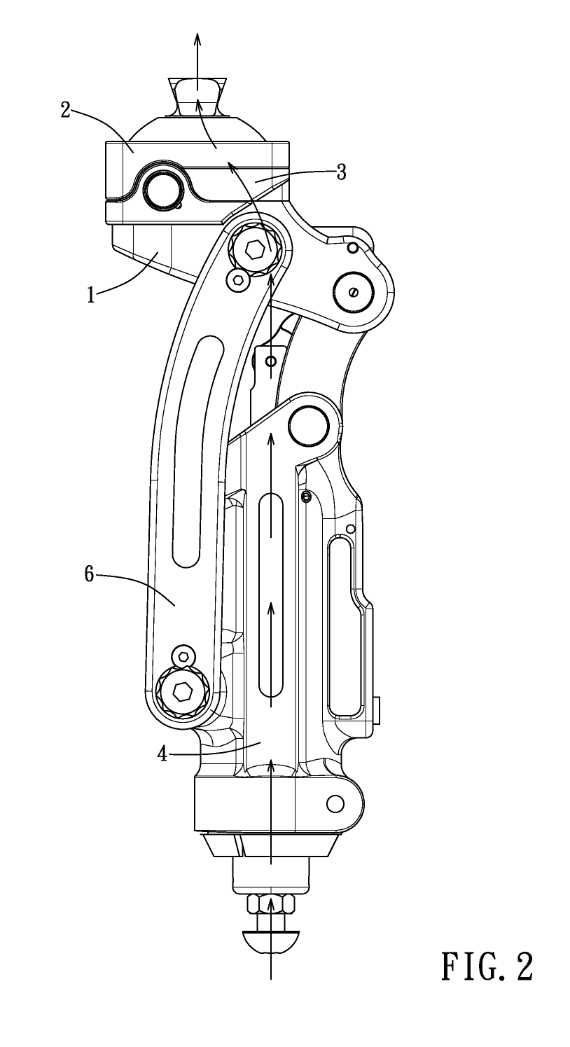

As shown in FIG. 2, the reaction force is transmitted on a transmission path from the prosthetic foot to the stump limb through the prosthetic lower leg 8, the connection seat 4, the knee member 1, the resilient member 3, the upper connecting member 2 and the prosthetic upper leg 7 in sequence.

For safety and stability concerns, the prosthetic foot, the prosthetic lower leg 8, the connection seat 4, and the knee member 1 may have a rigid and non-deformable structure and any relative displacement thereamong may not be preferred. Besides, the upper connecting member 2, the prosthetic upper leg 7 and the stump limb of the wearer are required to be stably connected in sequence and may have no relative pivotal movement or displacement thereamong. Hence, it is preferred to dispose the resilient member 3 between the knee member 1 and the upper connecting member 2. By way of such configuration, the reaction force transmitted from the knee member 1 can be partially absorbed by the resilient member 3 so as to alleviate discomfort of the wearer due to the reaction force. Additionally, even though the upper connecting member 2 is pivotally connected to the knee member 1, the resilient member 3 disposed therebetween tends to restrict the pivotal movement of the upper connecting member 2 relative to the knee member 1 to be within a limited range, and the upper connecting member 2 and the knee member 1 are fittingly connected without an applied force.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A knee joint for connection with a prosthetic upper leg and a prosthetic lower leg, said knee joint comprising:
a knee member;
an upper connecting member that is disposed on top of said knee member for connection with the prosthetic upper leg;
a resilient member disposed between said knee member and said upper connecting member; and
a connection seat having an upper portion pivotally connected to said knee member, and a lower portion for connecting with the prosthetic lower leg.

2. The knee joint as claimed in claim 1, wherein said upper connecting member is pivotally connected to said knee member, a bottom surface of said upper connecting member and an upper surface of said knee member cooperating to define a buffer space therebetween, said resilient member being disposed in said buffer space, said upper connecting member and said knee member compressing said resilient member when pivoting relative to each other.

3. The knee joint as claimed in claim 2, wherein said knee member has a pivot part disposed rearwardly of said buffer space and connected pivotally to said upper connecting member.

4. The knee joint as claimed in claim 2, wherein said resilient member is a rubber pad that is configured to conform to said bottom surface of the upper connecting member and said upper surface of said knee member, and is received fittingly in said buffer space.
